(19) United States
(12) Patent Application Publication
(10) Pub. No.: US 2008/0269914 A1
(43) Pub. Date: Oct. 30, 2008

(54) Direct contact moldable low temperature thermoplastic prosthetic devices

(75) Inventors: Daniel D. Coppens, Avondale, PA (US); John Damon Kirk, Ramsey, NJ (US); C. Eric Johnson, Baltimore, MD (US)

Correspondence Address: GOMEZ INTERNATIONAL PATENT OFFICE, LLC, 1501 N. RODNEY STREET, SUITE 101, WILMINGTON, DE, 19806, US

(73) Assignee: QFIX Systems, LLC, Avondale, PA (US)

(21) Appl. No.: 12/051,811

(22) Filed: Mar. 19, 2008

Related U.S. Application Data
(60) Provisional application No. 60/895,727, filed on Mar. 19, 2007.

(51) Int. Cl. A61F 2/60; A61F 2/80

(52) U.S. Cl. 623/33; 623/36

(57) ABSTRACT

The present invention provides direct contact moldable low temperature thermoplastic prosthetic devices and method of making same. A direct contact moldable low temperature thermoplastic prosthetic socket preform that is formable between 50° Celsius and 80° Celsius wherein the socket preform can be direct contact molded on at least one appendage of a patient thereby forming a prosthetic socket and wherein the prosthetic socket is reformable after initial forming. The present invention also provides method of creating a direct contact moldable and reformable prosthetic socket.

### CROSS REFERENCE TO RELATED APPLICATION

This application claims priority to and benefit of U.S. Provisional Application 60/895,727 filed 19 Mar. 2007, entitled Direct Contact Moldable Low Temperature Thermoplastic Prosthetic Devices.

### BACKGROUND OF THE INVENTION

The creation of prosthetic devices for upper and lower extremities generally requires the fabrication of a socket that slips over the limb. The shuttle, pylon and other components are mounted to the distal end of the socket. The comfort and functionality of the prosthetic device is largely dependent on the quality of the fit and properties of the materials used to fabricate the socket. For this reason, elaborate and time consuming methods have been developed to allow an accurate and comfortable socket to be fitted to the patient's limb. This often requires several steps in which a casting is taken from the limb, a negative is then made of the casting (this resembles the limb itself), and then a socket is molded over the negative. These sockets are commonly fabricated from high temperature thermoplastics. Several visits to the prosthetist are often required to obtain an adequate fit between the socket and limb.

Over time, these sockets wear and must be replaced. The patient may require several sockets over the life of the prosthetic device. The replacement socket is often made by taking an imprint of the inside of the existing socket. In this manner, a “copy of a copy” is created and, again, several fitting and adjustment sessions may be required to obtain a proper fit.

In addition, the limb may atrophy over time and require new prosthetic sockets to be made which fit the atrophied shape of the limb.

Low temperature thermoplastics have long been used to immobilize or position patients or patient body parts during or after various medical procedures. These medical procedures include, but are not limited to, radiotherapy patient immobilization, orthopedic casting or splinting, plastic and reconstructive surgery splinting, and orthotic or prosthetic socket cone production or reproduction. Aquaplast, a low temperature thermoplastic material invented by WFR/Aquaplast Corp and covered by U.S. Pat. No. 4,240,415 to Wartman, has been used to create impressions of limbs and preexisting sockets. U.S. Pat. No. 6,444,282 to Shirer describes a method for producing sockets impressions in this manner.

Many methods exist today to create prosthetic sockets. However, all of these techniques require ultimately that (1) an impression of the limb be made, (2) a model is produced which is a facsimile of the limb and then (3) a socket is produced and fitted to the limb.

It would be much preferred to have a method and materials from which the prosthetic socket could be produced directly on the limb (direct contact molding). This reduces the time, inconvenience and cost associated in fabricating prosthetic devices. By direct contact molding we mean that the socket is molded directly using the actual limb. This does not necessarily mean that the socket must come in contact with the limb during molding; a release agent, sock, slip sheet, Shrinkee Sleeve® or other separator may be placed between the limb and the socket during molding. However, the patient's limb is used directly as the form in the generation of the socket geometry.

### SUMMARY OF THE INVENTION

The present invention overcomes the above limitations of the prior art and provides direct contact moldable low temperature thermoplastic prosthetic devices. By creating a prosthetic socket directly on the limb of the patient you not only save time and cost but the socket of the present invention provides a better fit for the patient initially and over the life of the prosthetic since the prosthetic can be reformed.

Specifically, the present invention provides a direct contact moldable low temperature thermoplastic prosthetic socket preform that is formable between 50° Celsius and 80° Celsius wherein the socket preform can be direct contact molded on at least one appendage of a patient thereby forming a prosthetic socket and wherein the prosthetic socket is reformable after initial forming.

The instant invention also provides a method of creating a direct contact moldable and reformable prosthetic socket comprising; heating a socket preform until it is malleable; placing the preform directly over a patient's limb or limb facsimile; forming the prosthetic socket to the limb or limb facsimile; optionally trimming excess material; optionally smoothing rough edges; optionally applying an overlay to the socket; and optionally applying localized heat, to adjust the shape of the socket.

### BRIEF DESCRIPTION OF THE DRAWINGS

FIG. 1 illustrates a typical below the knee leg prosthetic device.

FIGS. 2A, 2B and 2C represent a conical socket preform of the present invention, the socket molded and trimmed, and the application of an overlay to the socket.

FIGS. 3A, 3B and 3C illustrate a sheet socket preform of the present invention, the socket molded, and a distal overlay applied to the socket.

FIGS. 4A and 4B illustrate the socket material of the present invention reinforced with a fiber layer and a multilayer construction.

FIG. 5 illustrates a socket preform with an integral reinforcing structure on the outside.

FIGS. 6A, 6B and 6C illustrate a socket preform, a molded socket and a molded socket with shuttle lock hardware.

FIG. 7 is a side view of a molded socket of the present invention.

FIG. 8 is a rear view of a molded socket of the present invention.

FIG. 9 shows typical shuttle lock hardware.

FIGS. 10A and 10B illustrate various socket overlays used with the present invention.

### DETAILED DESCRIPTION OF THE INVENTION

By creating new materials and methods it becomes possible to fabricate a prosthetic socket cone directly on the patient's limb, greatly reducing the time and expense required to create the prosthetic device. In addition, since the socket can be molded directly to the limb and easily reformed, a much better fit is achieved. Because the prosthetic is made from a low temperature thermoplastic, it can be reformed as the limb changes shape through swelling (edema) and with atrophy. By integrating the prosthetic hardware into the socket, the process of creating the prosthetic is further simplified. Sockets of the present invention are suitable for use in variety of prosthetic applications including below the knee, above the knee, below the elbow and above the elbow devices.

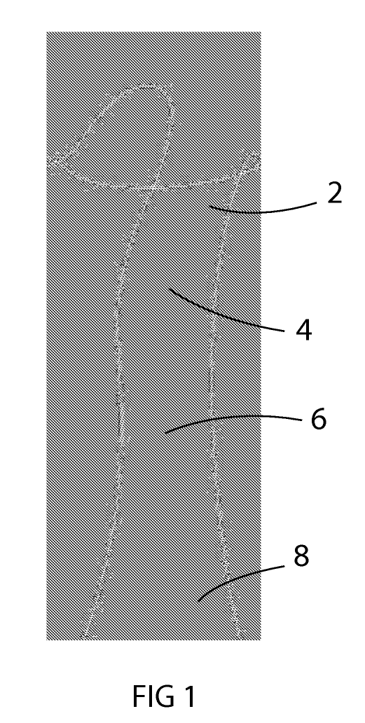

FIG. 1 illustrates a typical prosthetic device that is used for below the knee amputations. The main components of the device are; the prosthetic socket (2), the shuttle lock (4), the pylon (6) and the foot (8). Using the present invention, it is possible to direct mold the prosthetic socket (2) to the patient, customizing the fit and allowing the shape to be altered as the need arises.

As discussed above, current prosthetic sockets are produced in several steps because materials and methods have not existed that allow the socket to be molded directly on the patient. This limitation occurred for several reasons. High temperature thermoplastics cannot be directly molded on the patient because the molding temperatures are too high for patient contact. While low temperature thermoplastics lack the structural integrity required to perform service as the actual socket for the prosthetic device.

One method to create a direct prosthetic socket is to first create a socket preform from a reinforced or unreinforced polycaprolactone (PCL) low temperature thermoplastic. This preform can take the form of a precut sheet or a 3 dimensional molding. The PCL polymer is moldable at temperatures around 140° F., which coupled with PCL's unusual glass transition temperature characteristics, allows it to be formed directly against the skin without patient burning or discomfort. Typically the material is heated in a commercially available hot water bath. However, other methods such as heat guns, hot plates or ovens can also be used. In a preferred embodiment, the preformed socket cone is molded (typically by injection molding) to a conical shape which is close to the ultimate shape the socket will need to take to fit snuggly over the limb. Several standard sizes (small, medium, large, extra large, etc) are produced to adequately cover the general population. By reinforcing the PCL resin, adequate structural properties can be achieved to allow a prosthetic socket to be direct molded to the patient without further reinforcement. (See co-pending U.S. patent application Ser. No. 11/368,991 filed 6 Mar. 2006, titled Reinforced Low Temperature Thermoplastic Material, and hereby incorporated by reference in its entirety). This reinforced material has the structural integrity to accept the hardware required to create a prosthetic device and to retain its shape during use. Reinforcement of the PCL can be achieved by the method described in the patent application above, adding a variety of fillers such as talc, or by embedding reinforcing fabrics. Integrally (as opposed to applying a secondary overlay—see below) reinforcing the PCL socket cone is preferred because it removes the secondary overlay step and produces a lighter prosthetic device.

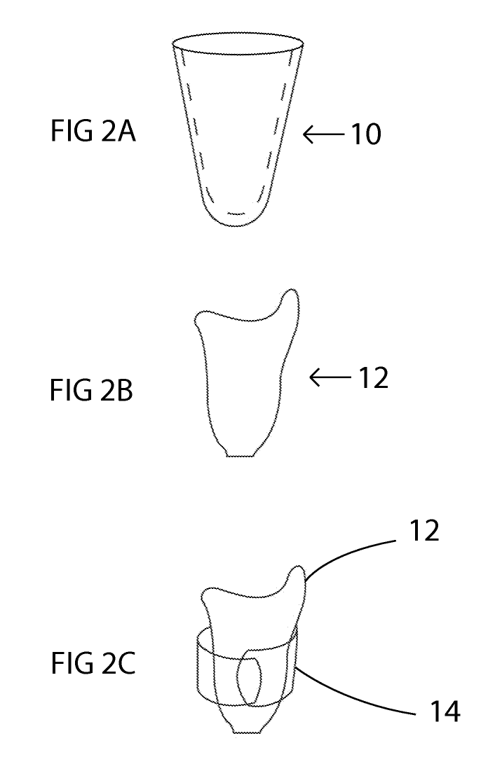

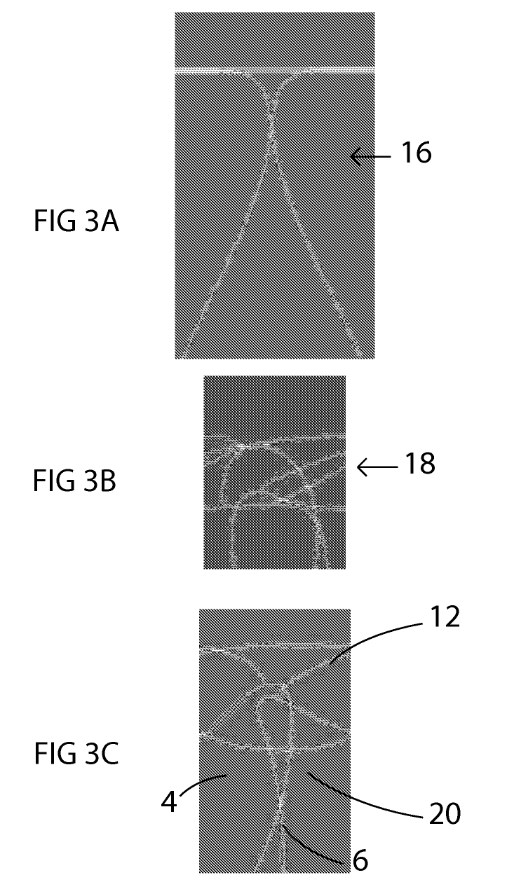

A socket preform (10), (16) is fabricated and then molded to the patient. FIGS. 2A-2C demonstrate the use of a conical socket preform (10), which is then molded and trimmed to for the socket (12). Optionally, an overlay (14) can be molded on to the socket to help reinforce the socket and/or to add aesthetic features such as artwork to the socket. In FIGS. 3A-3C, the preform (16) is comprised of a precut sheet of material. This is then folded and molded to the patient's limb to form the socket (18). FIG. 3C represents a socket (12) with shuttle lock (4) and pylon (6) hardware to which a distal overlay (20) has been applied. The distal overlay can be premolded to shape or preformed from sheet material.

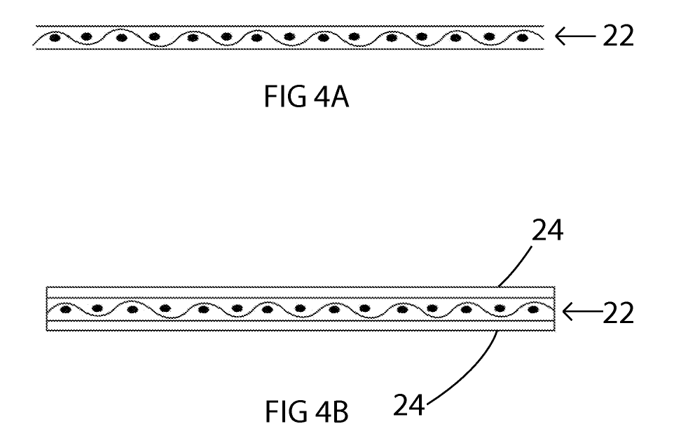

The socket material can be unreinforced or reinforced with a variety of filler materials. FIG. 4A represents the wall of a socket (22) that is reinforced with a fabric material. In FIG. 4B, the wall is comprised of a multilayer construction which in this case employs outer layers for improved patient comfort. These outer layers can be made from a variety of materials including, but not limited to, foam, PCL, a coating, and a fabric material.

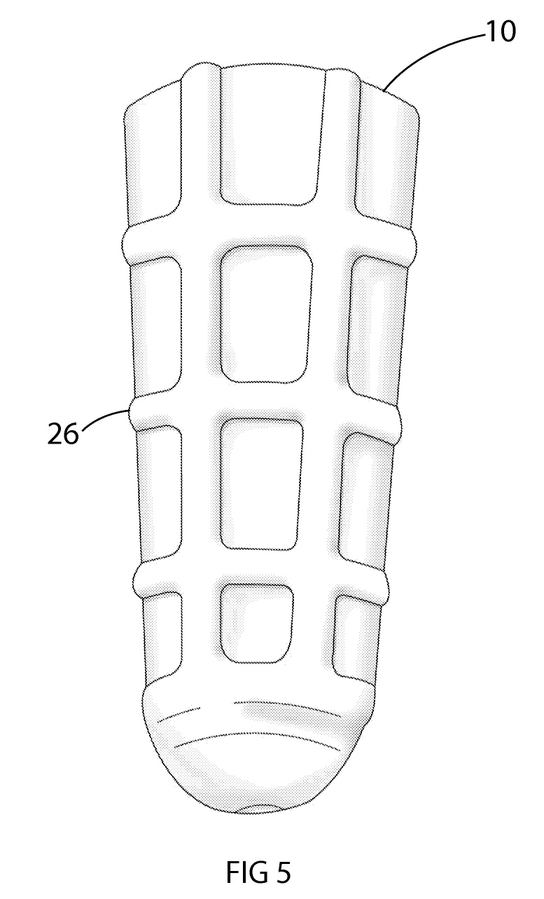

FIG. 5 illustrates a typical reinforcing geometry (26) that is suitable for this application. The reinforcing ribs may be co-molded in the preform or added secondarily as an overlay.

The polycaprolactone in the preform or socket can be reinforced with at least one selected from the group consisting of carbon fiber, aramid fiber, ultra high molecular weight polyethylene, fiberglass, woven fabric, non-woven fabric, fleece, knit fabric, cellulose, Nylon, polybenzoxazole (PBO), liquid crystal polymer fiber, talc, polypropylene, polyamide, polybutyleneterepthalate, man made fiber, cotton, wood pulp, natural fiber, silica, calcium silicate, cis-1,4 polydiolefin, ionomer, synthetic rubber, natural rubber, C. styrene-butadiene-styrene, glass spheres, glass micro balloons, phenolic spheres, phenolic micro balloons, plastic spheres, plastic micro balloons and styrene-isoprene-styrene triblock copolymer.

Structurally adequate prosthetic sockets can also be produced by creating an exterior structure, either integrally molded to the socket preform or through an overlay applied to the preform or formed socket. This reinforcement can take a variety of forms, including ribs, corrugation, etc. FIG. 5 shows a socket preform that contains raised axial and hoop ribs. These will stretch during forming of the prosthetic socket but continue to provide the required structural enhancement.

It is also possible to create sockets of adequate structural integrity by adding a secondary overlay. First the PCL socket is direct molded to the patient and then the overlay is applied to the outside of the PCL socket to increase structural integrity. Preferably this overlay is also produced from a PCL based thermoplastic but it is not required. Using a PCL allows the overlay to be molded in the same low temperature manner as the socket. The overlay locally increases the wall thickness of the socket thereby increasing its strength and stiffness. The overlay may itself be unreinforced or reinforced. Reinforcement can take many forms such as particulate, short fibers, long fibers, and fabric. By reinforcing the overlay, a thinner overlay may be used, reducing the overall weight of the prosthetic device. Another benefit of the overlay is that it may be used to enhance the appearance of the socket cone. By printing or embedding artwork in the overlay, designs, logos, camouflage and other effects can be produced. Since the prosthetic device must be worn by the patient long term, the benefits of enhanced aesthetic appearance are important to patient satisfaction. This is particularly true for pediatric cases. While it is critical to use an overlay if the initial socket is not reinforced, this does not restrict an overlay from being used with a reinforced socket cone.

Overlays can also be used at the distal end of the prosthetic device to reinforce the area of the hardware. In fact, the overlay may actually cover the hardware such as the shuttle. This adds to the structural integrity and helps finish the device, creating a definitive prosthetic device. A distal end overlay may take the shape of a pin wheel to make it easier to form over the end.

The mounting hardware can be attached to the socket before or after direct molding of the socket. In a preferred embodiment, the mounting hardware is co-molded to the PCL during injection molding. This provides a method of integrating the hardware into the socket.

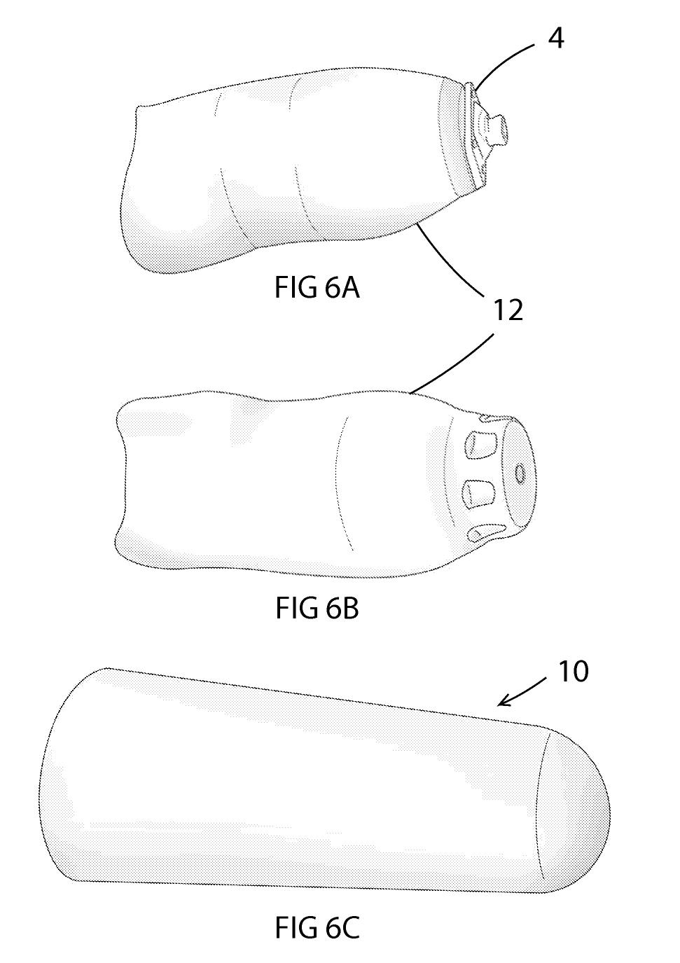

FIGS. 6A-6C show a socket preform (10), a molded socket (12) and a molded socket with shuttle lock hardware (4) installed. In a preferred embodiment, the shuttle lock hardware is co-molded in the socket preform.

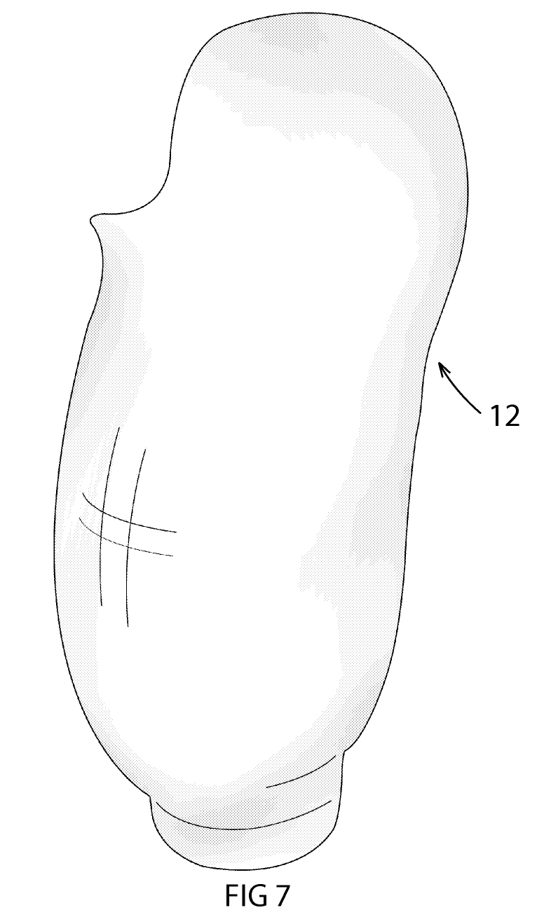

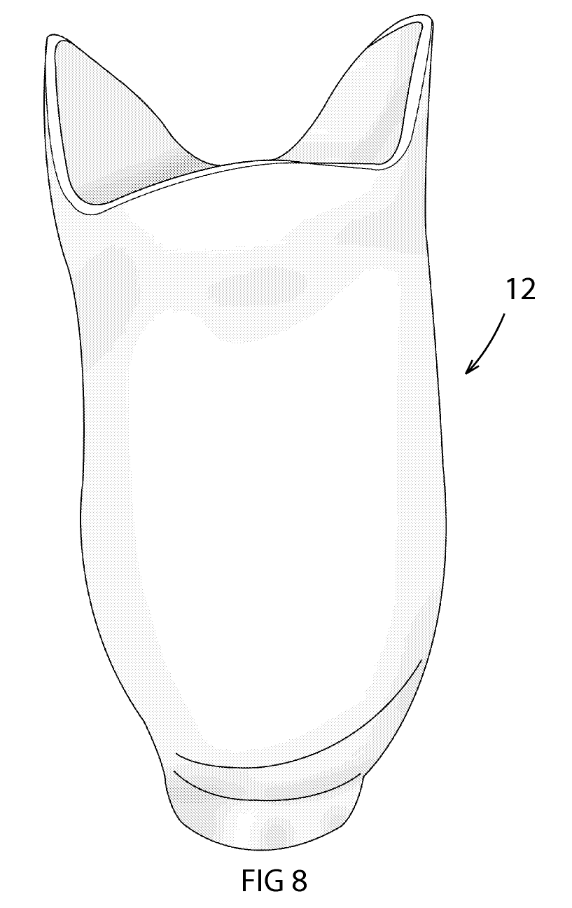

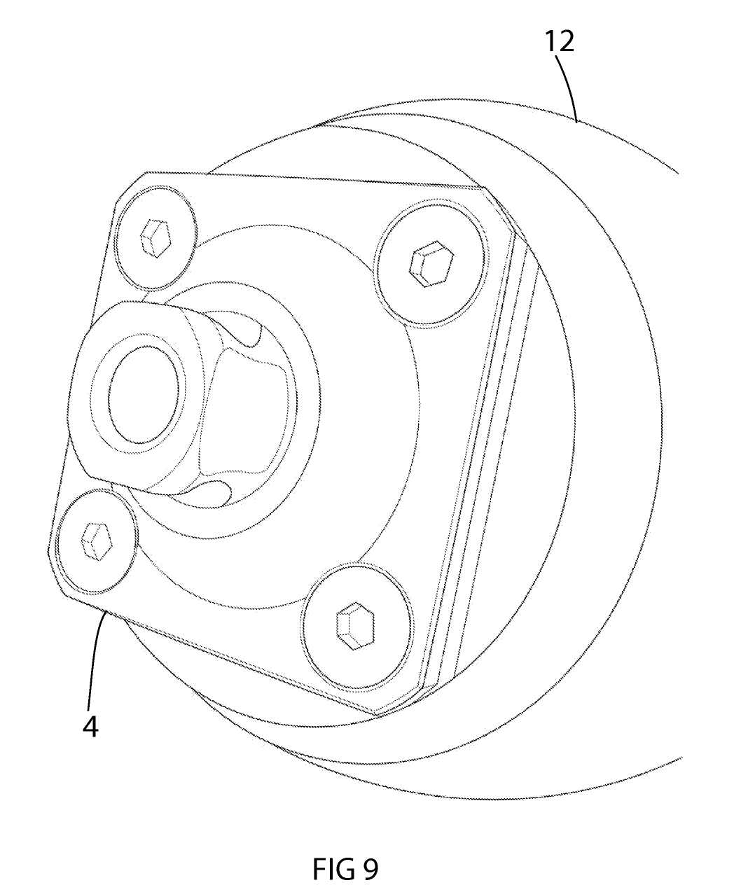

In FIGS. 7 and 8 various views of the molded socket (12) are shown. FIG. 9 shows a detailed illustration of typical shuttle lock (4) hardware.

Sockets of this invention can also be produced from sheet PCL material. A preform is cut from the sheet material. This can be accomplished with scissors, die cutting, or other methods. The preform is then heated, wrapped around the limb and seamed so that a prosthetic socket is formed. The process is aided by the fact that PCL sticks well to itself. A prosthetic of this kind may be created from either reinforced or unreinforced material. Overlays and hardware can be added as discussed above.

Sockets of this invention can be used as temporary prosthetics such as Dynamic test sockets which are commonly used for 4 to 6 months, they may be used a “check sockets” for determining fit, and they may be used as permanent definitive sockets. Because these sockets can be produced quickly and inexpensively, they are perfect for showering and bathing application where the patient may not want to expose their primary prosthetic device to a harsh environment.

It is often beneficial that the PCL thermoplastic be covered with a non-stick coating. This coating can be applied to the entire socket cone or to the inside surface only, leaving the outside free to more easily accept overlays. The comfort and performance of the socket can also be improved by creating a multi-layer preform. For example, a reinforced inner core of material may be used which has one or more layers of material applied to one or more of its surfaces so that the patient can not come in direct contact with the core material. This is particularly beneficial if the core material is reinforced with materials such as carbon fiber or fiberglass. These two materials can have a rough or “scratchy” feeling when rubbed in direct contact with the skin. This multi-layer effect can be achieved through co molding or co-extrusion.

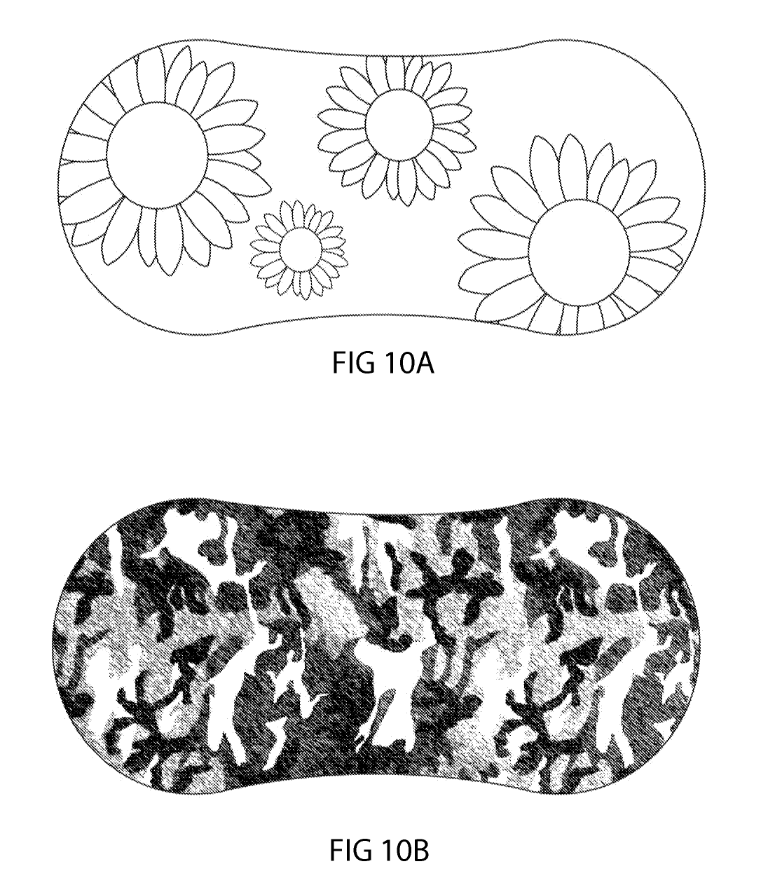

As stated before, overlays (14) may be used both to reinforce the socket and to add aesthetic value. Socket overlay does not need to be PCL based but can be high temperature thermoplastic, corrugated plastic sheet, carbon fiber Kevlar, fiberglass, woven and non-woven fabrics. FIGS 10A and 10B show two exemplary overlays (14). The overlays themselves may be made from reinforced or unreinforced material. FIG. 10B shows and overlay which consists of a camouflage fabric embedded in PCL resin. We have found that a variety of fabrics can work well, including but not limited to felts, denims and spandex. The felt materials expand upon heating to provide excellent structural reinforcement. The Spandex materials are very stretchy and easily conform to the molded socket shapes. All of these materials are commercially available with a variety of artwork.

It may also be beneficial to provide a foam or fabric inner layer to the PCL (polycaparolactone) thermoplastic in order to increase patient comfort. Knit, felt or other soft woven or non-woven fiber forms may be used to provide and inner layer that reduces chafing and may allow some airflow. A variety of open and closed cell foams exist that are beneficial for this purpose as well.

One of the primary benefits of this invention is that the socket may be reheated either locally or completely so that it may be reshaped. It can either be immersed in hot water or heated locally with a heat gun. Reforming is very hard to achieve with high temperature thermoplastics and impossible with thermosetting plastic socket systems. Typically after an amputation, the patient will experience edema or swelling which may subside over time. By reforming the socket, the patient can continue to have a comfortable fitting prosthesis throughout this period. Another example of a benefit that can be provided by reforming the socket is to create a patella bar. A patella bar is a ridge molded in to the socket just below the patella to help distribute a portion of the load to the patella region. This is extremely difficult to achieve with high temperature thermoplastic sockets but relatively simple with this invention.

Typically the wall thickness for sockets of this invention is between 1 mm and 10 mm. Overlays may be as thin as 0.1 mm and range up to 10 mm.

The present invention is further defined by the following claims.

We claim:

1) A direct contact moldable low temperature thermoplastic prosthetic socket preform that is formable between 50° Celsius and 80° Celsius wherein the socket preform can be direct contact molded on at least one appendage of a patient thereby forming a prosthetic socket and wherein the prosthetic socket is reformable after initial forming.

2) The socket preform of claim 1 comprising polycaprolactone thermoplastic.

3) The socket preform of claim 2 wherein the polycaprolactone is reinforced with at least one selected from the group consisting of carbon fiber, aramid fiber, ultra high molecular weight polyethylene, fiberglass, woven fabric, non-woven fabric, fleece, knit fabric, cellulose, Nylon, polybenzoxazole (PBO), liquid crystal polymer fiber, talc, polypropylene, polyamide, polybutyleneterepthalate, man made fiber, cotton, wood pulp, natural fiber, silica, calcium silicate, cis-1,4 polydiolefin, ionomer, synthetic rubber, natural rubber, C. styrene-butadiene-styrene, glass spheres, glass micro balloons, phenolic spheres, phenolic micro balloons, plastic spheres, plastic micro balloons and styrene-isoprene-styrene triblock copolymer.

4) The socket preform of claim 1 having a wall thickness from 1.0 mm to 10.0 mm.

5) The socket preform of claim 2 wherein the polycaprolactone is reinforced with at least one selected from the group consisting of a particulate, a fiber, a fine, a continuous fiber, and a fabric.

6) The socket preform of claim 2 wherein the polycaprolactone is cross-linked.

7) The socket preform of claim 3 wherein the filler comprises from 2% to 70% of the total material weight.

8) The formed prosthetic socket of claim 1 further comprising an reinforcement overlay.

9) The formed prosthetic socket of claim 8 wherein the overlay comprises polycaprolactone.

10) The formed prosthetic socket of claim 9 wherein the polycaprolactone is reinforced with at least one selected from the group consisting of a particulate, a fiber, a fine, a continuous fiber, and a fabric.

11) The formed prosthetic socket of claim 9 wherein the polycaprolactone is reinforced with at least one selected from the group consisting of carbon fiber, aramid fiber, ultra high molecular weight polyethylene, fiberglass, woven fabric, non-woven fabric, fleece, knit fabric, cellulose, Nylon, polybenzoxazole (PBO), liquid crystal polymer fiber, talc, polypropylene, polyamide, polybutyleneterepthalate, man made fiber, cotton, wood pulp, natural fiber, silica, calcium silicate, cis-1,4 polydiolefin, ionomer, synthetic rubber, natural rubber, C. styrene-butadiene-styrene, glass spheres, glass micro balloons, phenolic spheres, phenolic micro balloons, plastic spheres, plastic micro balloons and styrene-isoprene-styrene triblock copolymer.

12) The socket preform of claim 1 that is at least one shape selected from the group consisting of cylindrical, conical, and frustoconical.

13) The socket preform of claim 1 that is formed from sheet material.

14) The formed prosthetic socket of claim 1 wherein the socket or the overlay has a multi-layer construction.

15) The formed prosthetic socket of claim 8 wherein the socket or the overlay has multi-layer construction.

16) A prosthetic socket which can serve as at least one of a definitive and a dynamic socket.

17) The formed prosthetic socket of claim 1 further comprising external structural reinforcement molded integrally or applied secondarily.

18) The formed prosthetic socket of claim 11 wherein the filler comprises from 2% to 70% of the total material weight.

19) A method of creating a direct contact moldable and reformable prosthetic socket comprising;
a. heating a socket preform until it is malleable;
b. placing the preform directly over a patient's limb or limb facsimile;
c. forming the prosthetic socket to the limb or limb facsimile;
d. optionally trimming excess material;
e. optionally smoothing rough edges;
f. optionally applying an overlay to the socket; and
g. optionally applying localized heat, to adjust the shape of the socket.

20) The method of creating a direct contact moldable and reformable prosthetic socket of claim 19 further comprising placing at least one selected from the group consisting of a liner, a casting sock, a cast separator, and material over the limb prior to casting the prosthetic socket.

21) The method of creating a direct contact moldable and reformable prosthetic socket of claim 19 further comprising applying prosthetic mounting hardware or accessories to the socket preform prior to direct molding, wherein the mounting hardware is installed by at least one method selected from the group consisting of co-molding, securing with fasteners, and bonding.

22) The method of creating a direct contact moldable and reformable prosthetic socket of claim 19 further comprising applying prosthetic mounting hardware or accessories to the socket preform after direct molding.

23) The method of creating a direct contact moldable and reformable prosthetic socket of claim 19 using sheet material.

24) The method of creating a direct contact moldable and reformable prosthetic socket of claim 19 further comprising integrally molding or secondarily applying an external structural reinforcement.

25) The method of creating a direct contact moldable and reformable prosthetic socket of claim 19 further comprising placing an overlay at a distal end of the socket.
